(19) United States
(12) Patent Application Publication
(10) Pub. No.: US 2018/0207304 A1
(43) Pub. Date: Jul. 26, 2018

(54) MEDICAL CONDUIT

(71) Applicant: NIKKISO CO., LTD., Tokyo (JP)

(72) Inventor: Hideki ASANO, Tokyo (JP)

(21) Appl. No.: 15/827,939

(22) Filed: Nov. 30, 2017

(30) Foreign Application Priority Data
Jan. 20, 2017 (JP) 2017-008462

(51) Int. Cl. A61L 2/10; A61M 25/04

(52) U.S. Cl. CPC A61L 2/10; A61B 90/70; A61M 25/04

(57) ABSTRACT

A catheter which is one mode of a medical conduit of the present invention is placed inside a human body from the outside of the human body through human tissue. The catheter has a light introducing portion. The light introducing portion is provided on an outer wall surface of the catheter located on the outside-of-human-body side, and allows at least a part of ultraviolet light irradiated from the outside of the catheter to be totally reflected at an inner wall surface of the catheter toward the inside-of-human-body side.

### BACKGROUND OF THE INVENTION

The present invention relates to a catheter which is retained in, for example, the abdominal cavity.

There is disclosed a disinfection unit that sterilizes an area of a catheter retained in a patient that is inserted into human tissue (see the following Patent Literature 1). In the disinfection unit, a light source that performs surface emission of ultraviolet light is placed on a side surface that is more on the outer side than the area of the catheter inserted into the human tissue, and the light source irradiates ultraviolet light toward an insertion portion of the catheter.
[Patent Literature 1] JP2009-261957 A

### SUMMARY OF THE INVENTION

However, in the disinfection unit of the above-described Patent Literature 1, since UV that is surface-emitted from the outside of the patient transmits through a wall thickness portion of the catheter, there is a concern that sterilization against bacteria present between human tissue and a portion of the catheter inserted into the human tissue may be insufficient.

In view of this, the present invention provides a medical conduit that can increase the sterilizing effect against bacteria present between human tissue and a portion of the medical conduit surrounded by the human tissue.

To solve the problem, a medical conduit that is placed inside a human body from outside of the human body through human tissue, the medical conduit includes: a light introducing portion that is provided on an outer wall surface of the medical conduit located on an outside-of-human-body side and that allows at least a part of ultraviolet light to be totally reflected at an inner wall surface of the medical conduit toward an inside-of-human-body side, the ultraviolet light being irradiated from outside of the medical conduit.

The ultraviolet light that is totally reflected by the light introducing portion toward the inside-of-human-body side propagates toward the human tissue while being totally reflected between the outer wall surface and inner wall surface of the medical conduit. Portions of the inner wall surface of the medical conduit on the outside-of-human-body side and in the human tissue are both in contact with atmosphere, and thus, there is almost no difference in refractive index with the medical conduit. On the other hand, the outer wall surface of medical conduit is in contact with the human tissue instead of with atmosphere, and thus, the refractive index difference with the medical conduit is reduced. Thus, ultraviolet light that propagates between the outer wall surface and inner wall surface of the medical conduit located in the human tissue is easily emitted from the outer wall surface of the medical conduit.

As such, according to the medical conduit of the present invention, since the medical conduit itself serves as a waveguide for ultraviolet light, bacteria present between the medical conduit and the human tissue can be directly irradiated with ultraviolet light through the medical conduit being retained in the human body. In this manner, a medical conduit is implemented that can increase the sterilizing effect against bacteria present between human tissue and a portion of the medical conduit surrounded by the human tissue.

Note that it is preferred that the light introducing portion be a diffraction grating having projections and recesses which are provided at a predetermined period on the outer wall surface. In such a case, the period of the diffraction grating can be set according to the wavelength of ultraviolet light suitable for sterilizing effect, and thus, the sterilizing effect against bacteria present between human tissue and a portion of the medical conduit surrounded by the human tissue can be further increased.

It is preferred that the ratio of the period of the diffraction grating to the wavelength of the ultraviolet light be between 0.7 and 1.5 inclusive. In such a case, the range of entrance angles of ultraviolet light that enters the diffraction grating so as to be totally reflected at the inner wall surface of the medical conduit toward the inside-of-human-body side can be further widened. Accordingly, flexibility in the placement of a light source against the diffraction grating can be improved, and limitations on the shapes and types of light sources that can be used can be lessened.

It is preferred that the light introducing portion be a plurality of protrusions that protrude from the outer wall surface of the medical conduit, and the protrusions be provided with photorefractive surfaces that refract at least a part of the ultraviolet light toward the inside-of-human-body side. In such a case, even if the wavelength of ultraviolet light suitable for sterilizing effect is changed, at least a part of the ultraviolet light is refracted at the surfaces of the protrusions toward the inside-of-human-body side. Hence, even if the wavelength of ultraviolet light is changed, at least a part of the ultraviolet light is easily totally reflected at the inner wall surface of the medical conduit toward the inside-of-human-body side. Therefore, the versatility of the medical conduit can be improved.

It is preferable that the light introducing portion is a protrusion protruding from the outer wall surface so as to be farther away from a central axis in a longitudinal direction of the medical conduit as going toward the outside-of-human-body side from the inside-of-human-body side, and a light introducing surface through which the ultraviolet light is introduced is provided at an end on the outside-of-human-body side of the protrusion. In such a case, it makes it easy to visually and intuitively grasp the direction and position in which ultraviolet light is introduced. Therefore, when a medical professional or the like places a light source, misplacement can be reduced.

As described above, according to the present invention, a medical conduit can be provided that can increase the sterilizing effect against bacteria present between human tissue and a portion of the medical conduit surrounded by the human tissue.

### BRIEF DESCRIPTION OF THE DRAWINGS

FIG. 1 is a diagram showing a state in which a catheter is retained in a human body;

FIG. 2 is a diagram showing a part of the catheter of a first embodiment;

FIG. 3 is a diagram provided to describe diffraction of a diffraction grating;

FIG. 4 is a graph showing the results of simulation;

FIG. 5 is a graph showing the results of another simulation;

FIG. 6 is a diagram showing a part of a catheter of a second embodiment;

FIG. 7 is a diagram showing a light introducing portion of a shape different from that of the second embodiment;

FIG. 8 is a diagram showing a light introducing portion of a shape different from that of the second embodiment and that of FIG. 7;

FIG. 9 is a diagram showing a part of a catheter of a third embodiment; and

FIG. 10 is a diagram showing a part of a catheter of a fourth embodiment.

### DETAILED DESCRIPTION OF THE INVENTION

Embodiments for carrying out the present invention and variants thereof will be illustrated below together with the accompanying drawings. The embodiments and variants thereof which will be illustrated below are provided for easy understanding of the present invention and should not to be construed as limiting the present invention. In addition, the present invention may be changed without departing from the spirit and scope thereof.

### (1) First Embodiment

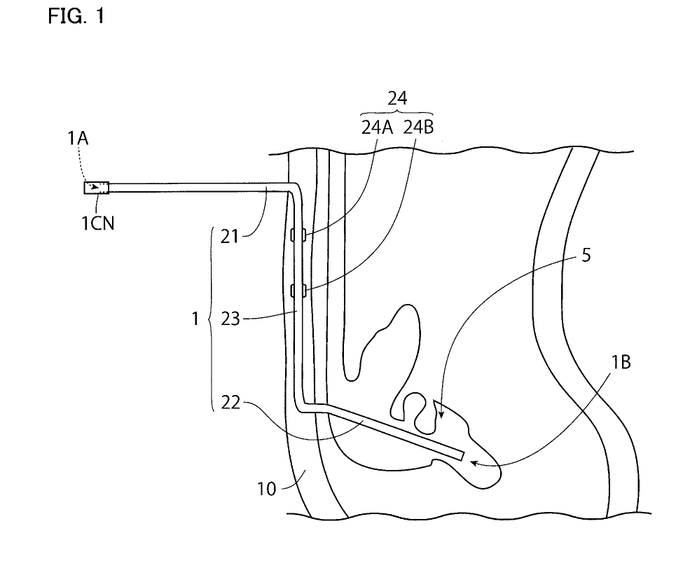

FIG. 1 is a diagram showing a state in which a catheter is retained in a human body. As shown in FIG. 1, a catheter 1 of the present embodiment is a medical conduit used as a flow passage that carries dialysate into and out of the peritoneal cavity. The catheter 1 is placed inside the human body from the outside of the human body through human tissue 10.

The catheter 1 has an outside-of-human-body placement portion 21, an inside-of-human-body placement portion 22, and a human tissue placement portion 23. The outside-of-human-body placement portion 21 is a portion located outside the human body, the inside-of-human-body placement portion 22 is a portion located in the peritoneal cavity which is the inside of the human body, and the human tissue placement portion 23 is a portion located within the human tissue 10. Note that the human tissue placement portion 23 is a portion buried under the skin and thus is also called a subcutaneous tunnel.

An open end 1A of the outside-of-human-body placement portion 21 is covered with a catheter connector 1CN. An open end 13 of the inside-of-human-body placement portion is placed in a recess portion 5 of the peritoneal cavity. The catheter connector 1CN is configured to be connectable to connecting parts such as a cock and a tube in a replaceable manner. Note that the recess portion 5 of the peritoneal cavity is a dent portion of the peritoneal cavity and is called the pouch of Douglas. The recess portion 5 is located between the bladder and rectum in males and between the uterus and rectum in females.

The human tissue placement portion 23 is provided with cuffs 24. The cuffs 24 are fibrous members provided on a surface of the human tissue placement portion 23. The cuffs 24 are fixed to the human tissue 10 by adhesion to the human tissue 10, and thereby suppress unintentional dislodgement of the catheter 1. In an example shown in FIG. 1, the cuffs 24 include two cuffs, an outer-side cuff 24A and an inner-side cuff 243. The outer-side cuff 24A is located nearer to the open end 1A on the outside-of-human-body side of the catheter 1. The inner-side cuff 243 is located nearer to the open end 13 on the inside-of-human-body side of the catheter 1. Note that the number of the cuffs 24 may be one or three or more, or the cuffs 24 may be omitted.

Such a catheter 1 has an inside diameter in a range of 2.0 mm to 4.0 mm inclusive, and has a wall thickness in a range of 0.5 mm to 1.5 mm inclusive. Note that the wall thickness of the catheter 1 is the difference between the outside diameter and inside diameter of the catheter 1. Note also that materials of the catheter 1 include, for example, a silicone resin. Additives such as an antimicrobial agent, an antioxidant, and a slip additive may be contained. Note that in the present embodiment it is preferred that additives that absorb ultraviolet light not be contained.

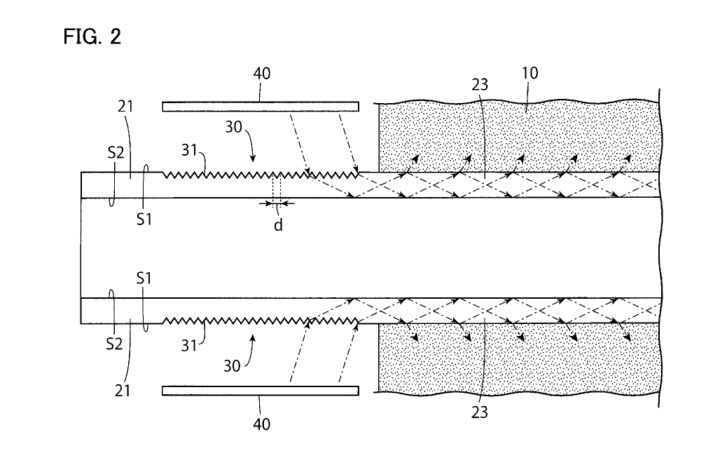

FIG. 2 is a diagram showing a part of the catheter of the first embodiment. As shown in FIG. 2, the catheter 1 has a light introducing portion 30 that allows at least a part of ultraviolet light which is irradiated from the outside of the catheter 1 to be totally reflected at an inner wall surface S2 of the catheter 1 toward the inside-of-human-body side. The light introducing portion 30 is provided on an outer wall surface S1 of the outside-of-human-body placement portion 21.

Note that it is desirable to provide the light introducing portion 30 in a range of 3 mm to 30 mm inclusive from an exit site which is an area where the catheter 1 comes out of the human tissue 10.

In the case of the present embodiment, the light introducing portion 30 is a diffraction grating 31 having projections and recesses which are provided at a predetermined period d on the outer wall surface S1 of the catheter 1. In an example shown in FIG. 2, the projections and recesses are provided on the outer wall surface S1 by forming recess portions that are recessed from the outer wall surface S1. Note, however, that projections and recesses may be provided on the outer wall surface S1 by forming projection portions that project from the outer wall surface S1.

In addition, in the present embodiment, the outside shape of the diffraction grating 31 at a cross section along a longitudinal direction of the catheter 1 is a triangle wave-shaped pattern in which an isosceles portion of an isosceles triangle is repeated. It is preferred that the period d of the diffraction grating 31 be defined by a relationship with the wavelength of ultraviolet light entering the diffraction grating 31. Specifically, it is preferred that the ratio of the period d of the diffraction grating 31 to the wavelength of ultraviolet light entering the diffraction grating 31 be between 0.7 and 1.5 inclusive. For example, when the wavelength of ultraviolet light is 310 nm, the period d of the diffraction grating 31 is between 220 nm and 450 nm inclusive. In addition, when the wavelength of ultraviolet light is 280 nm, the period d of the diffraction grating 31 is between 200 nm and 420 nm inclusive. The wavelength of ultraviolet light is, for example, in a range of 270 nm to 340 nm inclusive, taking into account sterilization capability, the influence on the human body, etc.

When sterilization is performed using the catheter 1 of the present embodiment, for example, a light-emitting device 40 is placed in a circumferential direction of the catheter 1 with a predetermined distance provided from the light introducing portion 30 of the catheter 1 retained in the human body. As the light-emitting device 40, for example, a light emitting diode (LED), a laser diode (LD), or an organic electroluminescence (EL) that performs surface emission of ultraviolet light having a peak in a band of 270 nm to 340 nm is used. Note that the light-emitting device 40 does not need to perform surface emission.

When the light introducing portion 30 is irradiated with ultraviolet light emitted from the light-emitting device 40, at least a part of the ultraviolet light is diffracted by the diffraction grating 31 and is thereby totally reflected at the inner wall surface S2 of the catheter 1 toward the inside-of-human-body side. At the outside-of-human-body placement portion 21, the totally reflected ultraviolet light propagates toward the human tissue placement portion 23 while being totally reflected between the outer wall surface S1 and inner wall surface S2 of the catheter 1.

At the human tissue placement portion 23, the inner wall surface S2 of the human tissue placement portion 23 is in contact with atmosphere as with the outside-of-human-body placement portion 21, and thus, the refractive index difference between the catheter 1 and the inner side of the inner wall surface S2 is large. On the other hand, the outer wall surface S1 of the human tissue placement portion 23 is in contact with the human tissue 10 instead of with atmosphere, and thus, the refractive index difference between the catheter 1 and the human tissue 10 is reduced. Thus, at least a part of the ultraviolet light that propagates while being totally reflected at the human tissue placement portion 23 is radiated toward the human tissue 10 from the outer wall surface S1 of the human tissue placement portion 23. Therefore, when bacteria are present between the human tissue placement portion 23 and the human tissue 10, the bacteria are directly irradiated with the ultraviolet light.

For example, when ultraviolet light on the order of 1 mW per 1×1-cm square area is radiated from the human tissue placement portion 23, if the peak wavelength emitted from the light-emitting device 40 is 265 nm, then the sterilization rate is 99% with the irradiation time on the order of roughly four seconds. In addition, if the peak wavelength emitted from the light-emitting device 40 is 320 nm, then the sterilization rate is 99% with the irradiation time on the order of roughly 50 minutes.

As described above, the catheter 1 of the present embodiment has, on the outer wall surface S1 of the outside-of-human-body placement portion 21, the light introducing portion 30 that allows at least a part of ultraviolet light to be totally reflected at the inner wall surface S2 of the catheter 1. Hence, as described above, the catheter 1 itself serves as a waveguide for ultraviolet light. Therefore, bacteria present between the human tissue placement portion 23 of the catheter 1 and the human tissue 10 can be directly irradiated with ultraviolet light through the catheter 1 being retained in the human body. In this manner, the catheter 1 is implemented that can increase the sterilizing effect against bacteria present between the human tissue 10 and the human tissue placement portion 23 surrounded by the human tissue 10.

In addition, in the case of the present embodiment, the light introducing portion 30 is the diffraction grating 31 having projections and recesses which are provided at the predetermined period d on the outer wall surface S1 of the catheter 1. Hence, the period d of the diffraction grating 31 can be set according to the wavelength of ultraviolet light suitable for sterilizing effect. Therefore, the sterilizing effect against bacteria present between the human tissue 10 and the human tissue placement portion 23 surrounded by the human tissue 10 can be further increased.

Note that, as described above, it is preferred that the ratio of the period d of the diffraction grating 31 to the wavelength of ultraviolet light entering the diffraction grating 31 be between 0.7 and 1.5 inclusive. By doing so, the range of entrance angles of ultraviolet light that enters the diffraction grating 31 so as to be totally reflected at the inner wall surface S2 of the catheter 1 toward the inside-of-human-body side can be widened. Accordingly, flexibility in the placement of the light-emitting device 40 against the diffraction grating 31 can be improved, and limitations on the shapes and types of the light-emitting device 40 that can be used can be lessened.

When the ratio of the period d of the diffraction grating 31 to the wavelength of ultraviolet light entering the diffraction grating 31 is between 0.9 and 1.13 inclusive, the period d of the diffraction grating 31 is comparable with the wavelength of the ultraviolet light. In this case, the range of entrance angles of ultraviolet light that enters the diffraction grating 31 so as to be totally reflected at the inner wall surface S2 of the catheter 1 toward the inside-of-human-body side can be further widened.

Here, simulation is performed for the range of entrance angles at which ultraviolet light that is totally reflected at the inner wall surface S2 of the catheter 1 toward the inside-of-human-body side enters the diffraction grating 31.

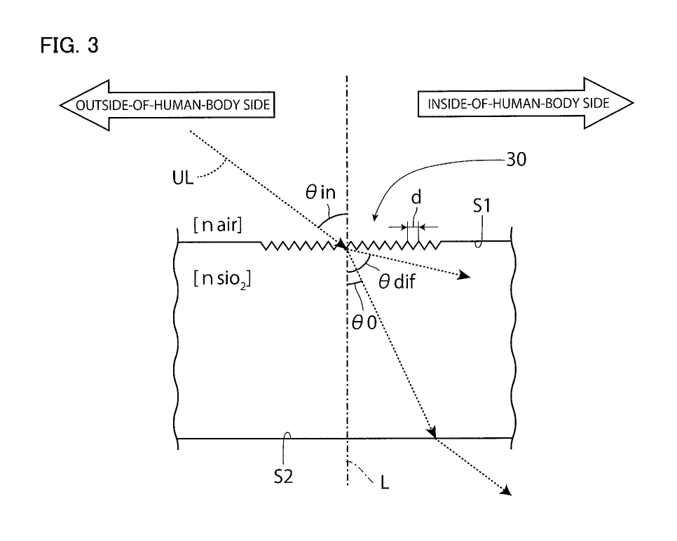

In the simulation, as shown in FIG. 3, when the refractive index of the catheter 1 is nsio₂, the refractive index of atmosphere is nair, the period of the diffraction grating 31 is d, and the wavelength of ultraviolet light UL entering the diffraction grating 31 is λ, the diffraction angle θm of the mth order is computed by the following equation:

nsio₂·sin θm=m˜λ/d (1)

In addition, in the simulation, it is premised that when the angle of refraction is θ₀, Snell's law of the following equation holds true:

nair·sin θₐᵢᵣ=nsio₂·sin θ₀ (2)

When the refractive index nsio₂ of the catheter 1 is 1.46, the refractive index nair of atmosphere is 1, and the wavelength λ of the ultraviolet light UL is 310 nm in the above-described equations (1) and (2), the critical angle at which negative first-order light is totally reflected is 46 degrees. Note that the negative first-order light is first-order diffracted light which is diffracted toward the inside-of-human-body side.

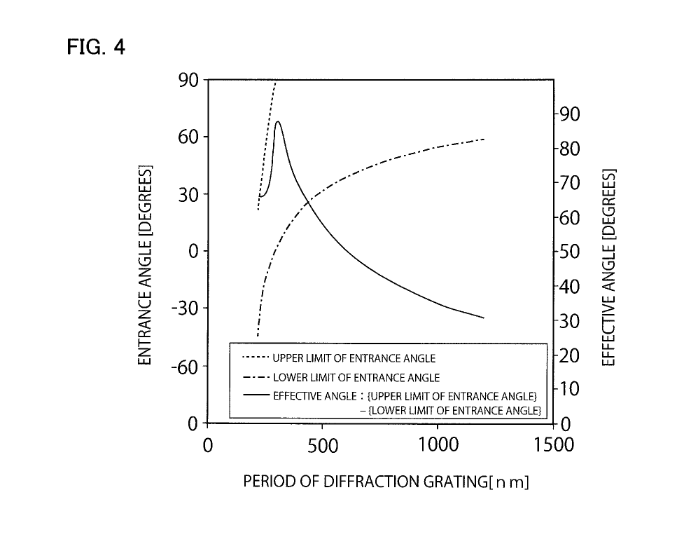

FIG. 4 shows the results of obtaining, based on such simulation, a range of entrance angles θin of ultraviolet light at which the angle θdif of negative first-order light that is introduced into the catheter 1 is greater than a critical angle of 46 degrees. Note that the angle θdif is an angle formed by the normal line L to the outer wall surface S1 of the catheter 1 and the negative first-order light.

A dotted line and a dash-dotted line shown in FIG. 4 respectively indicate the upper and lower limits of the entrance angle at which the angle θdif formed by the normal line L and the negative first-order light is greater than the critical angle when an angle formed by the normal line L to the outer wall surface S1 of the catheter 1 and the ultraviolet light UL entering the diffraction grating 31 from the outside-of-human-body side with reference to the normal line L is the entrance angle θin (FIG. 3).

Furthermore, effective angles shown in FIG. 4 each are an angle obtained by subtracting a lower limit of the entrance angle from an upper limit of the entrance angle. If the entrance angle is in a range of those effective angles, the angle θdif formed by the negative first-order light and the normal line L is greater than the critical angle. For example, when the upper limit of the entrance angle is +30 degrees and the lower limit of the entrance angle is −40 degrees, if the entrance angle is in a range of 30 degrees on the positive side to 40 degrees on the negative side with reference to the normal line L, the angle θdif formed by the normal line L and the negative first-order light is greater than the critical angle.

As shown in FIG. 4, diffraction does not occur with the period d of the diffraction grating 31 being shorter than 220 nm. In addition, when the period d of the diffraction grating 31 is 300 nm which is substantially the same length as the wavelength “310 nm” of the ultraviolet light UL, the upper limit of the entrance angle is 90 degrees and the lower limit of the entrance angle is 2 degrees. In this case, the effective angle is 88 degrees which is largest, and if the entrance angle is in a range of 90 degrees to 2 degrees on the positive side with reference to the normal line L, the angle θdif formed by the normal line L and the negative first-order light is greater than a critical angle of 46 degrees. That is, it can be seen that almost whatever the entrance angle of the ultraviolet light UL entering the inside-of-human-body side from the outside-of-human-body side, the ultraviolet light UL can be totally reflected at the inner wall surface S2 of the catheter 1 toward the inside-of-human-body side.

Furthermore, in a range of a period d of the diffraction grating 31 of 220 nm to 450 nm inclusive, an effective angle of 60 degrees or more is secured. It can be seen that in this range the ratio of the period d of the diffraction grating 31 to the wavelength of the ultraviolet light UL is between 0.71 and 1.45 inclusive, and the entrance angle of the ultraviolet light UL that enters the diffraction grating 31 so as to be totally reflected at the inner wall surface S2 of the catheter 1 toward the inside-of-human-body side is widened.

Furthermore, in a range of a period d of the diffraction grating 31 of 280 nm to 350 nm inclusive, an effective angle of 70 degrees or more is secured. It can be seen that in this range the ratio of the period d of the diffraction grating 31 to the wavelength of the ultraviolet light UL is between 0.9 and 1.13 inclusive, and the entrance angle of the ultraviolet light UL that enters the diffraction grating 31 so as to be totally reflected at the inner wall surface S2 of the catheter 1 toward the inside-of-human-body side is further widened.

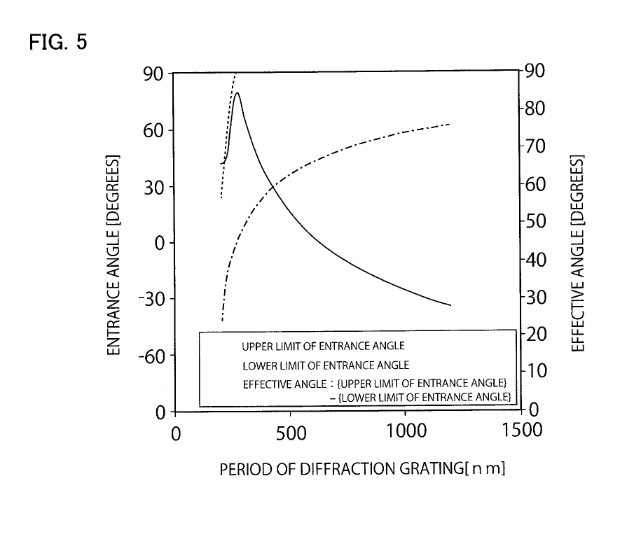

Note that FIG. 5 shows the results of obtaining a range of entrance angles θin of ultraviolet light UL at which the angle θdif formed by the normal line L and the negative first-order light is greater than the critical angle for a case in which the wavelength λ of the ultraviolet light UL in the above-described simulation is changed to 280 nm and other parameters have the same conditions as those of the above-described simulation.

As shown in FIG. 5, when the period d of the diffraction grating 31 is 280 nm which is the same length as the wavelength “280 nm” of the ultraviolet light UL, the effective angle is 85 degrees which is largest. In addition, in a range of a period d of the diffraction grating 31 of 200 nm to 420 nm inclusive, an effective angle of 60 degrees or more can be secured. It can be seen that in this range the ratio of the period d of the diffraction grating 31 to the wavelength of the ultraviolet light UL is between 0.71 and 1.5 inclusive, and the entrance angle of the ultraviolet light UL that enters the diffraction grating 31 so as to be totally reflected at the inner wall surface S2 of the catheter 1 toward the inside-of-human-body side is widened. Furthermore, in a range of a period d of the diffraction grating 31 of 260 nm to 300 nm inclusive, an effective angle of 80 degrees or more can be secured. It can be seen that in this range the ratio of the period d of the diffraction grating 31 to the wavelength of the ultraviolet light UL is between 0.93 and 1.07 inclusive, and the entrance angle of the ultraviolet light UL that enters the diffraction grating 31 so as to be totally reflected at the inner wall surface S2 of the catheter 1 toward the inside-of-human-body side is further widened.

As such, even if the wavelength λ of the ultraviolet light UL is changed, when the period d of the diffraction grating 31 is comparable with the wavelength λ of the ultraviolet light UL, the effective angle is further increased. In addition, it has been found that even if the wavelength λ of the ultraviolet light UL is reduced, if the period d of the diffraction grating 31 is comparable with that wavelength λ, then almost whatever the entrance angle of the ultraviolet light UL entering the inside-of-human-body side from the outside-of-human-body side, the ultraviolet light UL can be totally reflected at the inner wall surface S2 of the catheter 1 toward the inside-of-human-body side.

Note that, in the above-described first embodiment, the outside shape of the diffraction grating 31 at the cross section along the longitudinal direction of the catheter 1 is a triangle wave-shaped pattern in which an isosceles portion of an isosceles triangle is repeated. However, the outside shape of the diffraction grating 31 at the cross section along the longitudinal direction of the catheter 1 may be any other projection and recess shaped pattern.

### (2) Second Embodiment

Next, a second embodiment will be described. Note that the same configurations as those described in the first embodiment are denoted by the same reference signs and overlapping description is omitted unless otherwise particularly described.

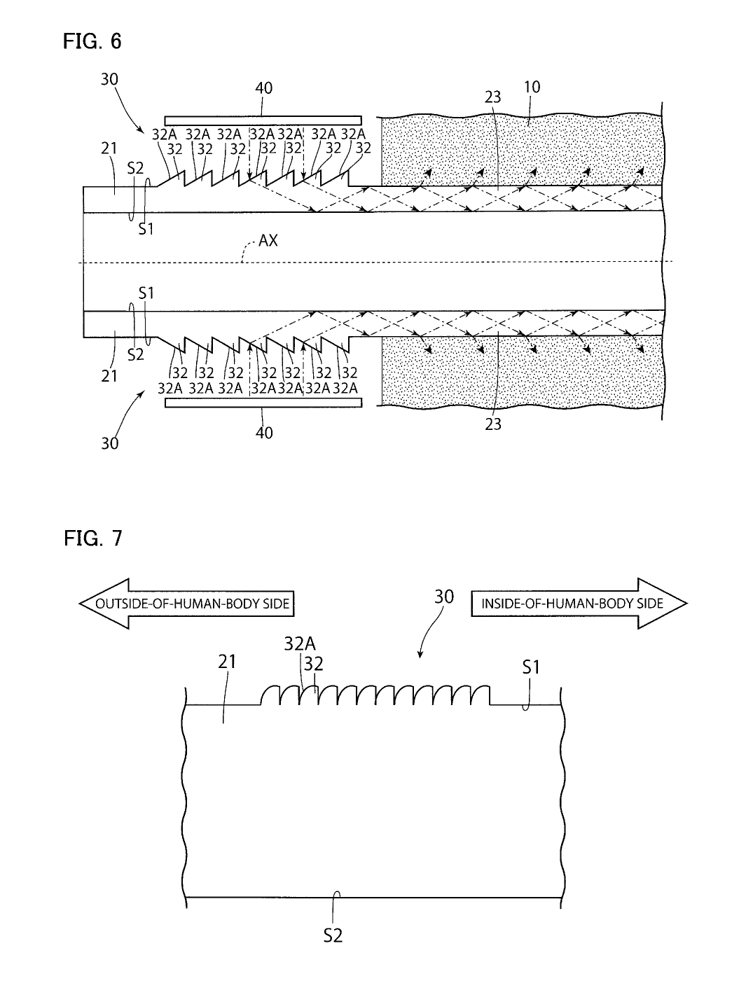

FIG. 6 is a diagram showing a part of a catheter 1 of the second embodiment. As shown in FIG. 6, a light introducing portion 30 of the present embodiment is a plurality of protrusions 32 that protrude from an outer wall surface S1 of the catheter 1. The outside shape of each protrusion 32 at a cross section along a longitudinal direction of the catheter 1 is a sawtooth shape having a hypotenuse of a right angle and another side that forms an acute angle with the hypotenuse. The hypotenuses of the sawtooth-shaped protrusions 32 are placed on the outside-of-human-body side. At least a part of ultraviolet light is refracted at the hypotenuses toward the inside-of-human-body side. Namely, each sawtooth-shaped protrusion 32 is provided with a photorefractive surface 32A that refracts at least a part of ultraviolet light toward the inside-of-human-body side. The photorefractive surface 32A is a flat surface that is inclined farther away from a central axis AX in the longitudinal direction of the catheter 1 as going toward the inside-of-human-body side from the outside-of-human-body side.

Note that although, in an example shown in FIG. 6, the outside shapes of the protrusions 32 at the cross section along the longitudinal direction of the catheter 1 are sawtooth shapes of the same shape and the same size, at least either one of the shape and size of some or all of the protrusions 32 may be different.

When ultraviolet light is emitted to such sawtooth-shaped protrusions 32 from a light-emitting device 40, at least a part of the ultraviolet light is refracted at the photorefractive surfaces 32A of the protrusions 32 toward the inside-of-human-body side and is totally reflected at an inner wall surface S2 of the catheter 1. At an outside-of-human-body placement portion 21, as in the above-described first embodiment, the totally reflected ultraviolet light propagates toward a human tissue placement portion 23 while being totally reflected between the outer wall surface S1 and inner wall surface S2 of the catheter 1.

In addition, as in the above-described first embodiment, the outer wall surface S1 of the human tissue placement portion 23 is in contact with human tissue 10 instead of with atmosphere, and thus, the refractive index difference decreases. Thus, at least a part of the ultraviolet light that propagates while being totally reflected at the human tissue placement portion 23 is radiated toward the human tissue 10 from the outer wall surface S1 of the human tissue placement portion 23. Therefore, when bacteria are present between the human tissue placement portion 23 and the human tissue 10, the bacteria are directly irradiated with the ultraviolet light.

As described above, the light introducing portion 30 of the present embodiment is the plurality of protrusions protruding from the outer wall surface S1 of the catheter 1, and the protrusions 32 have the photorefractive surfaces 32A that refract at least a part of ultraviolet light toward the inside-of-human-body side.

In such a light introducing portion 30, even if the wavelength of ultraviolet light suitable for sterilizing effect is changed, at least a part of the ultraviolet light is refracted at the photorefractive surfaces 32A toward the inside-of-human-body side. Hence, even if the wavelength of ultraviolet light is changed, at least a part of the ultraviolet light is easily totally reflected at an inner wall surface of the catheter 1 toward the inside-of-human-body side. Therefore, the versatility of the catheter 1 can be improved.

Note that, in the second embodiment, the outside shape of each protrusion 32 at the cross section along the longitudinal direction of the catheter 1 is a sawtooth shape having a hypotenuse of a right angle and another side that forms an acute angle with the hypotenuse. However, the outside shape of the protrusions 32 at the cross section along the longitudinal direction of the catheter 1 may be any other shape.

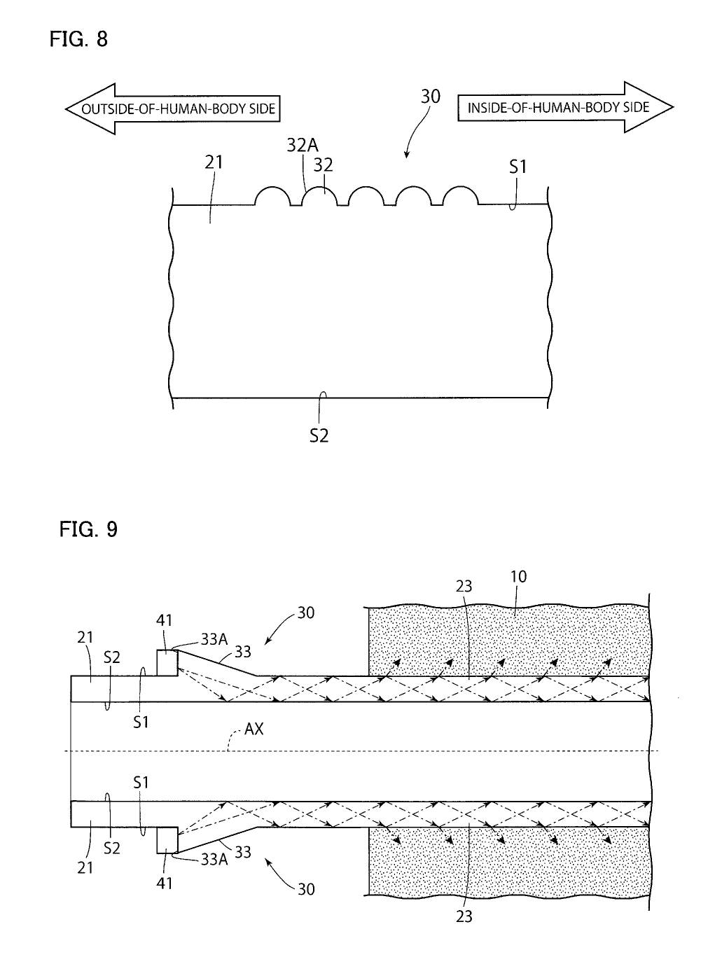

For example, although the photorefractive surfaces 32A of the protrusions 32 are flat surfaces in the example shown in FIG. 6, as shown in FIG. 7, the photorefractive surfaces 32A may be projected, curved surfaces. In addition, for example, as shown in FIG. 8, the outside shape of the protrusions 32 at the cross section along the longitudinal direction of the catheter 1 may be semicircular. Note that when the outside shape of the protrusions 32 is semicircular, at least a part of a surface of each protrusion 32 serves as a photorefractive surface 32A that refracts at least a part of ultraviolet light toward the inside-of-human-body side.

### (3) Third Embodiment

Next, a third embodiment will be described. Note that the same configurations as those described in the first embodiment are denoted by the same reference signs and overlapping description is omitted unless otherwise particularly described.

FIG. 9 is a diagram showing a part of a catheter 1 of the third embodiment. As shown in FIG. 9, a light introducing portion 30 of the present embodiment is a truncated cone-shaped protrusion 33 that protrudes from an outer wall surface S1 so as to be farther away from a central axis AX in a longitudinal direction of the catheter 1 as going toward the outside-of-human-body side from the inside-of-human-body side.

The radius on the inside-of-human-body side of the truncated cone-shaped protrusion 33 is approximately the same as that of the outside shape of the catheter 1, and the radius on the outside-of-human-body side is larger than the radius on the inside-of-human-body side.

In addition, a light introducing surface 33A through which ultraviolet light is introduced is provided at an end on the outside-of-human-body side of the truncated cone-shaped protrusion 33. In an example shown in FIG. 9, the light introducing surface 33A is parallel to a plane orthogonal to the central axis AX in the longitudinal direction of the catheter 1.

In the case of the present embodiment, for example, a ring-shaped light-emitting device 41 that emits ultraviolet light having a peak in a band of 270 nm to 340 nm is placed such that a light-emitting surface of the light-emitting device 41 is in contact with the light introducing surface 33A of the catheter 1 retained in the human body. Note that the light-emitting device 41 may be placed with a predetermined distance provided between the light-emitting surface of the light-emitting device 41 and the light introducing surface 33A. When the light-emitting surface of the light-emitting device 41 is distanced from the light introducing surface 33A, a member that reduces a refractive index difference with the protrusion 33 over a refractive index difference between air and the protrusion 33 may be interposed between the light-emitting surface and the light introducing surface 33A.

When ultraviolet light is emitted from the light-emitting device 41, at least a part of the ultraviolet light enters the light introducing surface 33A of the truncated cone-shaped protrusion 33 and is totally reflected at an inner wall surface S2 of the catheter 1 toward the inside-of-human-body side. At an outside-of-human-body placement portion 21, as in the above-described first embodiment, the totally reflected ultraviolet light propagates toward a human tissue placement portion 23 while being totally reflected between the outer wall surface S1 and inner wall surface S2 of the catheter 1.

In addition, as in the above-described first embodiment, the outer wall surface S1 of the human tissue placement portion 23 is in contact with human tissue 10 instead of with atmosphere, and thus, the refractive index difference decreases. Thus, at least a part of the ultraviolet light that propagates while being totally reflected at the human tissue placement portion 23 is radiated toward the human tissue 10 from the outer wall surface S1 of the human tissue placement portion 23. Therefore, when bacteria are present between the human tissue placement portion 23 and the human tissue 10, the bacteria are directly irradiated with the ultraviolet light.

As described above, the light introducing portion 30 of the present embodiment is the truncated cone-shaped protrusion 33 that protrudes from the outer wall surface S1 so as to be farther away from the central axis AX in the longitudinal direction of the catheter 1 as going toward the outside-of-human-body side from the inside-of-human-body side. In addition, the light introducing surface 33A through which ultraviolet light is introduced is provided at the end on the outside-of-human-body side of the truncated cone-shaped protrusion 33.

Such a light introducing portion 30 makes it easy to visually and intuitively grasp the direction and position in which ultraviolet light is introduced. Therefore, when a medical professional or the like places a light source, misplacement can be reduced.

### (4) Fourth Embodiment

Next, a fourth embodiment will be described. Note that the same configurations as those described in the first embodiment are denoted by the same reference signs and overlapping description is omitted unless otherwise particularly described.

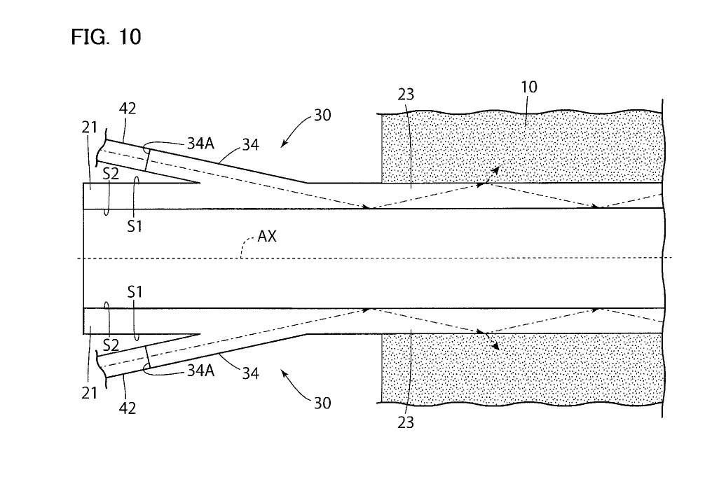

FIG. 10 is a diagram showing a part of a catheter 1 of the fourth embodiment. As shown in FIG. 10, a light introducing portion 30 of the present embodiment is cylindrical protrusions 34 that protrude from an outer wall surface S1 so as to be farther away from a central axis AX in a longitudinal direction of the catheter 1 as going toward the outside-of-human-body side from the inside-of-human-body side.

The plurality of cylindrical protrusions 34 are provided and placed at predetermined intervals in a circumferential direction of the outer wall surface S1 of an outside-of-human-body placement portion 21.

In addition, a light introducing surface 34A through which ultraviolet light is introduced is provided at an end on the outside-of-human-body side of each cylindrical protrusion 34. In an example shown in FIG. 10, the light introducing surfaces 34A are inclined with respect to the central axis AX in the longitudinal direction of the catheter 1.

In the case of the present embodiment, for example, end surfaces of optical fibers 42 abut on the light introducing surfaces 34A, respectively, and ultraviolet light having a peak in a band of 270 nm to 340 nm is emitted from the end surfaces of the optical fibers 42.

At least a part of the ultraviolet light emitted from the end surfaces of the optical fibers 42 enters the corresponding light introducing surfaces 34A of the cylindrical protrusions 34, propagates along the cylindrical protrusions 34, and is totally reflected at an inner wall surface S2 of the catheter 1 toward the inside-of-human-body side. At the outside-of-human-body placement portion 21, as in the above-described first embodiment, the totally reflected ultraviolet light propagates toward a human tissue placement portion 23 while being totally reflected between the outer wall surface S1 and inner wall surface S2 of the catheter 1.

In addition, as in the above-described first embodiment, the outer wall surface S1 of the human tissue placement portion 23 is in contact with human tissue 10 instead of with atmosphere, and thus, the refractive index difference decreases. Thus, at least a part of the ultraviolet light that propagates while being totally reflected at the human tissue placement portion 23 is radiated toward the human tissue 10 from the outer wall surface S1 of the human tissue placement portion 23. Therefore, when bacteria are present between the human tissue placement portion 23 and the human tissue 10, the bacteria are directly irradiated with the ultraviolet light.

As described above, the light introducing portion 30 of the present embodiment is the cylindrical protrusions 34 that protrude from the outer wall surface S1 so as to be farther away from the central axis AX in the longitudinal direction of the catheter 1 as going toward the outside-of-human-body side from the inside-of-human-body side. In addition, the light introducing surfaces 34A through which ultraviolet light is introduced are provided at the respective ends on the outside-of-human-body side of the cylindrical protrusions 34.

As in the above-described third embodiment, such a light introducing portion 30 makes it easy to visually and intuitively grasp the direction and position in which ultraviolet light is introduced. Therefore, when a medical professional or the like places a light source, misplacement can be reduced.

In addition, the cylindrical protrusions 34 of the present embodiment can selectively allow ultraviolet light that is totally reflected at the inner wall surface S2 of the catheter 1 toward the inside-of-human-body side to be introduced through the light introducing surfaces 34A, and can allow the ultraviolet light to propagate to the inner wall surface S2 of the catheter 1.

### (5) Variants

Although the above-described embodiments are described above as examples, the embodiments may be modified. For example, in the above-described embodiments, a catheter 1 for peritoneal dialysis is applied as a medical conduit. However, other catheters, e.g., a urinary catheter, are applicable as medical conduits. In addition, a drain may be applied as a medical conduit.

1. A medical conduit that is placed inside a human body from outside of the human body through human tissue, the medical conduit comprising:
a light introducing portion that is provided on an outer wall surface of the medical conduit located on an outside-of-human-body side and that allows at least a part of ultraviolet light to be totally reflected at an inner wall surface of the medical conduit toward an inside-of-human-body side, the ultraviolet light being irradiated from outside of the medical conduit.

2. The medical conduit according to claim 1, wherein the light introducing portion is a diffraction grating having projections and recesses, the projections and recesses being provided on the outer wall surface at a predetermined period in a longitudinal direction of the medical conduit and in a direction intersecting the longitudinal direction.

3. The medical conduit according to claim 2, wherein a ratio of the period of the diffraction grating to a wavelength of the ultraviolet light is between 0.7 and 1.5 inclusive.

4. The medical conduit according to claim 1, wherein the light introducing portion is a plurality of protrusions protruding from the outer wall surface of the medical conduit, and each of the protrusions is provided with a photorefractive surface that refracts at least a part of the ultraviolet light toward the inside-of-human-body side.

5. The medical conduit according to claim 1, wherein the light introducing portion is a protrusion protruding from the outer wall surface so as to be farther away from a central axis in a longitudinal direction of the medical conduit as going toward the outside-of-human-body side from the inside-of-human-body side, and a light introducing surface through which the ultraviolet light is introduced is provided at an end on the outside-of-human-body side of the protrusion.
